(19) United States
(12) Patent Application Publication
(10) Pub. No.: US 2007/0100372 A1
(43) Pub. Date: May 3, 2007

(54) Embolic protection device having a filter

(75) Inventor: Darin G. Schaeffer, Bloomington, IN (US)

Correspondence Address: BRINKS HOFER GILSON & LIONE/CHICAGO/COOK, PO BOX 10395, CHICAGO, IL, 60610, US

(73) Assignee: COOK INCORPORATED, BLOOMINGTON, IN (US)

(21) Appl. No.: 11/591,837

(22) Filed: Nov. 2, 2006

Related U.S. Application Data
(60) Provisional application No. 60/732,883, filed on Nov. 2, 2005.

(51) Int. Cl. A61M 29/00

(52) U.S. Cl. 606/200

(57) ABSTRACT

An embolic protection device for capturing emboli during treatment of a stenotic lesion in a body vessel is disclosed. In one example, the device comprises a filter and a filter portion made of an extracellular matrix circumferentially attached to the filter. The filter comprises a plurality of struts having first ends attached together at a center portion along a longitudinal axis. Each strut has an arcuate segment extending from the first end to a second end. The struts are configured to move between an expanded state for engaging with the body vessel and a collapsed state for filter retrieval or delivery. The filter portion is configured for allowing blood to flow therethrough and for capturing emboli when the filter is in the expanded state.

### CROSS-REFERENCE TO RELATED APPLICATIONS

This application claims the benefit of U.S. Provisional Application No. 60/732,883 filed on Nov. 2, 2005, entitled “EMBOLIC PROTECTION DEVICE HAVING A FILTER,” the entire contents of which are incorporated herein by reference.

### BACKGROUND OF THE INVENTION

The present invention relates to medical devices. In particular, the present invention relates to embolic protection devices for capturing emboli during treatment of a stenotic lesion in a body vessel.

Embolic protection to capture emboli within the vasculature is a growing concern in the medical industry. Currently, there are a number of approaches for embolic protection to prevent emboli from traveling within the vasculature to create an undesirable embolism, e.g., pulmonary embolism. For example, filters are more commonly being used for trapping emboli in the filter to prevent pulmonary embolism. Also, anti-platelet agents and anticoagulants may be used to breakdown blood clots. Moreover, snares and baskets (e.g., stone retrieval baskets) are more commonly used for retrieving urinary calculi. Additionally, occlusion coils are commonly used to occlude aneurysms and accumulate thrombi in a body vessel.

Treatments for a stenotic lesion provide a potential in releasing blood clots and other thrombi plaque in the vasculature of the patient. One example is the treatment for a carotid artery stenosis. Generally, carotid artery stenosis is the narrowing of the carotid arteries, the main arteries in the neck that supply blood to the brain. Carotid artery stenosis (also called carotid artery disease) is a relatively high risk factor for ischemic stroke. The narrowing is usually caused by plaque build-up in the carotid artery. Plaque forms when cholesterol, fat and other substances form in the inner lining of an artery. This formation process is called atherosclerosis.

Depending on the degree of stenosis and the patient's overall condition, carotid artery stenosis has been treated with surgery. The procedure (with its inherent risks) is called carotid endarterectomy, which removes the plaque from the arterial walls. Carotid endarterectomy has proven to benefit patients with arteries substantially narrowed, e.g., by about 70% or more. For people with less narrowed arteries, e.g., less than about 50%, an anti-clotting drug may be prescribed to reduce the risk of ischemic stroke. Examples of these drugs are anti-platelet agents and anticoagulants.

Carotid angioplasty is a more recently developed treatment for carotid artery stenosis. This treatment uses balloons and/or stents to open a narrowed artery. Carotid angioplasty is a procedure that can be performed via a standard percutaneous transfemoral approach with the patient anesthetized using light intravenous sedation. At the stenosis area, an angioplasty balloon is delivered to predilate the stenosis in preparation for stent placement. The balloon is then removed and exchanged via catheter for a stent delivery device. Once in position, a stent is deployed across the stenotic area. If needed, an additional balloon can be placed inside the deployed stent for post-dilation to make sure the struts of the of the stent are pressed firmly against the inner surface of the vessel wall.

During the stenosis procedure however, there is a risk of such blood clots and thrombi being undesirably released into the blood flow within the vasculature. Embolic or distal protection devices have been implemented to capture emboli from a stenotic lesion undergoing angioplasty. However, many current embolic protection devices restrict flow when deployed within the vasculature of the patient. Moreover, many embolic protection devices are relatively difficult to collapse and retrieve after the need for such device in the vasculature passes.

Thus, there is a need to provide a device and method for distally protecting and capturing emboli within a body lumen during a stenosis procedure, without relatively restricting flow and with relatively easy retrievability.

### BRIEF SUMMARY OF THE INVENTION

The present invention generally provides an embolic protection device that minimizes restricted flow when deployed within the vasculature of a patient and that is relatively easy to retrieve.

In one embodiment, the present invention provides an embolic protection device for capturing emboli during treatment of a stenotic lesion in a body vessel. The device comprises a filter and filter portion circumferentially attached to the filter. The filter comprises a plurality of struts having first ends attached together at a center portion along a longitudinal axis. Each strut has an arcuate segment extending from the first end to a second end. The struts are configured to move between an expanded state for engaging with the body vessel and a collapsed state for filter retrieval or delivery. The filter portion is circumferentially attached to the filter at each of the second ends. The filter portion is configured for allowing blood to flow therethrough and for capturing emboli when the filter is in the expanded state.

In another embodiment, the present invention provides an embolic protection assembly for capturing emboli during treatment of a stenotic lesion in a body vessel. The assembly comprises a balloon catheter having a tubular body portion and an expandable balloon attached to and in fluid communication with the tubular body portion for angioplasty at the stenotic lesion. The expandable balloon has distal and proximal portions. The assembly further comprises the emboli protection device coaxially disposed within the catheter during treatment of the stenotic lesion in the body vessel.

In another example, the present invention provides a method for embolic protection during treatment of stenotic lesion in a body vessel. The method comprises percutaneously introducing the balloon catheter in the body vessel and deploying the embolic protection device in a deployed state downstream from the stenotic lesion to capture emboli during treatment of the stenotic lesion.

In yet another example, the embolic capture device comprises a ring attached to the second ends and is configured to expand between the expanded and collapsed states. The filter portion is circumferentially attached to the ring. The filter portion is configured for allowing blood to flow therethrough and for capturing emboli when the filter is in the expanded state.

Further objects, features, and advantages of the present invention will become apparent from consideration of the following description and the appended claims when taken in connection with the accompanying drawings.

### BRIEF DESCRIPTION OF THE DRAWINGS

FIG. 1 is an environment view of an embolic capture device having a filter in an expanded state in accordance with one embodiment of the present invention;

FIG. 2 is a perspective side view of the embolic capture device in FIG. 1;

FIG. 3 is another environmental view of the device in the expanded state;

FIG. 4 is a side perspective view of the device in the expanded state;

FIG. 5 is a side view of the device in a collapsed state within a delivery member;

FIG. 6a is a side view of an embolic capture assembly for capturing emboli during treatment of a stenotic lesion in a body vessel in accordance with one embodiment of the present invention;

FIG. 6b is an exploded view of the assembly in FIG. 6a;

FIG. 7 is a flow chart of one method for capturing emboli during treatment of a stenotic lesion in a body vessel; and

FIGS. 8 and 9 are side views of an embolic capture device in accordance with another embodiment of the present invention.

### DETAILED DESCRIPTION OF THE INVENTION

The present invention generally provides an embolic capture device that minimizes restricted flow when deployed within the vasculature of a patient and that is relatively easy to retrieve after the risk of releasing blood clots and thrombi within the vasculature has passed. Embodiments of the present invention generally provide an embolic protection device comprising a filter including a plurality of struts having first ends attached together along a longitudinal axis. In one example, the device further comprises a filter portion made of an extracellular matrix and that is circumferentially attached to the filter at each of the second ends. When deployed in the body vessel, the filter portion opens to an expanded state of the device allowing blood to flow therethrough for capturing emboli. The struts of the filter allow for relatively easy removal from the body vessel. This may be accomplished by distally threading a catheter over the struts until the filter is collapsed within the catheter.

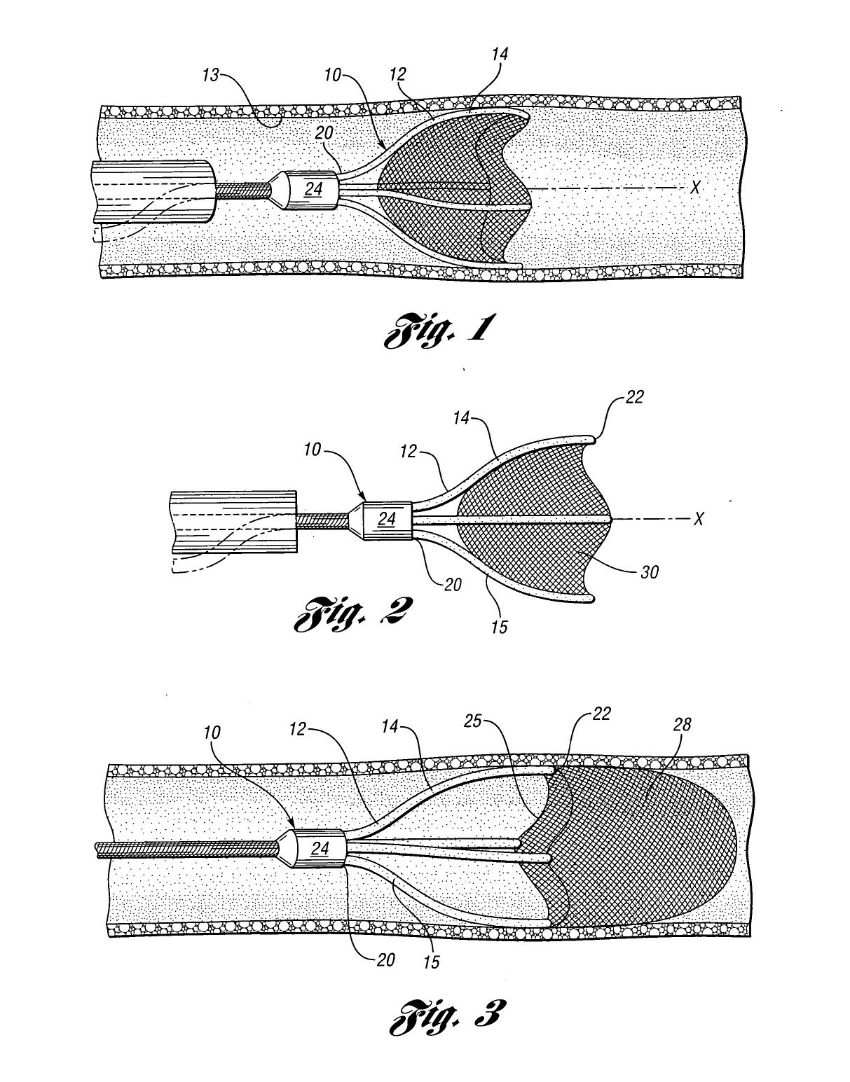

FIG. 1 illustrates an embolic protection device 10 for capturing emboli during treatment of a stenotic lesion in a body vessel in accordance with one embodiment of the present invention. As shown in FIGS. 1 and 2, the device 10 comprises a filter 12 including a plurality of struts 14 having first ends 20 attached together at a center portion along a longitudinal axis X. In this embodiment, each strut 14 has an arcuate segment 15 extending from the first end 20 to a second end 22. The arcuate segment 15 may take on any arcuate shape as it extends between the first and second ends.

As shown, each arcuate segment 15 has a soft S-shape. Each arcuate segment 15 is formed with a first curved portion that is configured to softly bend away from the longitudinal or central axis of the filter 12 and a second curved portion that is configured to softly bend toward the longitudinal axis of the filter. Due to the soft bends of each arcuate segment 15, a prominence or a point of inflection on the strut 14 is substantially avoided to aid in non-traumatically engaging the vessel wall. In the expanded state, each arcuate segment 15 extends arcuately along a longitudinal axis and linearly relative to a radial axis from the first end 14 to the anchoring ends. In this embodiment, the struts 14 extend linearly relative to the radial axis and avoid entanglement with other struts.

The struts 14 preferably are configured to move between an expanded state for engaging the body vessel and a collapsed state for filter 12 retrieval or delivery. In this embodiment, the filter 12 in the expanded state comprises four primary struts 14. As shown, the first ends 20 of the struts 14 emanate from a hub 24 that attaches the struts 14 together at the center point. In this embodiment, the struts 14 are preferably formed from wire having a round cross-section. Of course, it is not necessary that the struts 14 have a round or near round cross-section. For example, the struts 14 could take on any shape with arcuate edges to maintain non-turbulent blood flow therethrough.

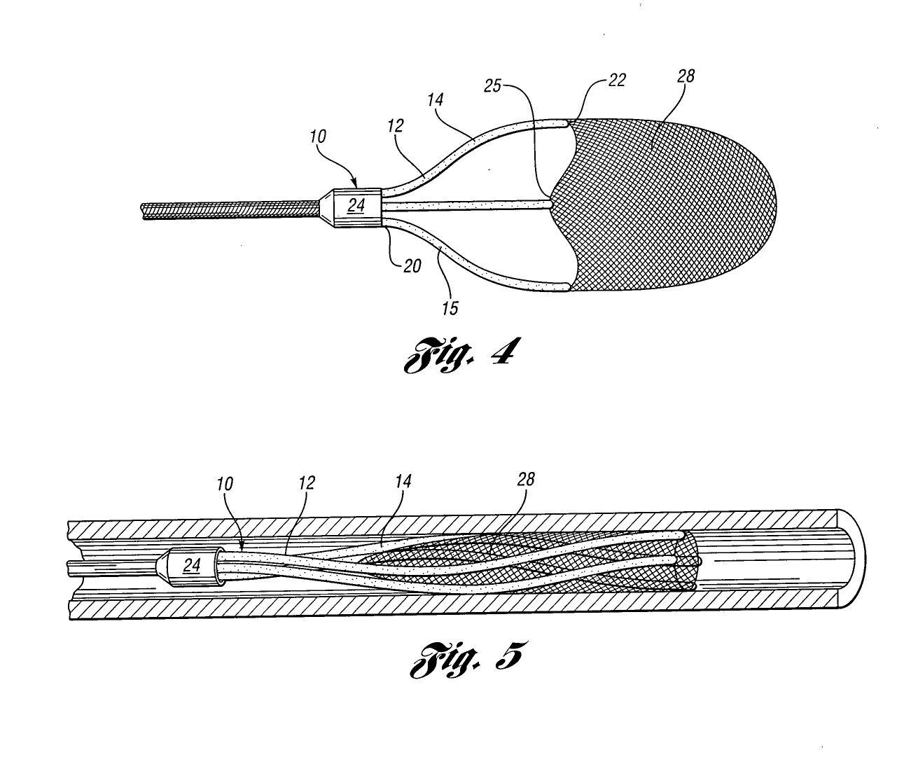

As shown in FIGS. 3 and 4, each of the struts 14 terminates at the second or anchoring end 22. Each of the arcuate segments 15 and the anchoring ends 22 will engage the vessel wall when the filter 12 is deployed at a delivery location in the blood vessel. As shown, the struts 14 are configured to move between a collapsed state for filter delivery and retrieval and an expanded state for engaging the blood vessel and capturing emboli during angioplasty. The filter 12 preferably extends longitudinally as shown in FIG. 4, defining the longitudinal axis of the filter 12. The filter 12 further radially expands and collapses, defining the radial axis of the filter 12. In this embodiment, the hub 24 houses and attaches the first ends 20 of the struts 14. The first ends 20 attach at the center point in the hub 24.

Although the embodiments of this device 10 have been disclosed as being constructed from wire having a round cross section, it could also be cut from a tube of suitable material by laser cutting, electrical discharge machining or any other suitable process.

FIGS. 3 and 4 illustrate a filter portion 28, e.g., an extracellular matrix portion. In this embodiment, the filter portion 28 is circumferentially attached to the filter 12 at each of the second ends 22. The filter portion 28 is configured for allowing blood to flow therethrough and for capturing emboli when the filter 12 is in the expanded state. As shown, the filter portion 28 includes a lip 25 attached to each of the second ends 22 defining an opening of the filter portion when the filter 12 is in the expanded state. The lip 25 extends to a closed end for capturing emboli.

The filter portion 28 may be comprised of any suitable material to be used for capturing emboli from the stenotic lesion during treatment thereof. In one embodiment, the filter portion 28 is made of connective tissue material for capturing emboli. In this embodiment, the connective tissue comprises extracellular matrix (ECM). As known, ECM is a complex structural entity surrounding and supporting cells that are found within mammalian tissues. More specifically, ECM comprises structural proteins (e.g., collagen and elastin), specialized protein (e.g., fibrillin, fibronectin, and laminin), and proteoglycans, a protein core to which are attached are long chains of repeating disaccharide units termed of glycosaminoglycans.

Most preferably, the extracellular matrix is comprised of small intestinal submucosa (SIS). As known, SIS is a resorbable, acellular, naturally occurring tissue matrix composed of ECM proteins and various growth factors. SIS is derived from the porcine jejunum and functions as a remodeling bioscaffold for tissue repair. SIS has characteristics of an ideal tissue engineered biomaterial and can act as a bioscaffold for remodeling of many body tissues including skin, body wall, musculoskeletal structure, urinary bladder, and also supports new blood vessel growth. In many aspects, SIS is used to induce site-specific remodeling of both organs and tissues depending on the site of implantation. In theory, host cells are stimulated to proliferate and differentiate into site-specific connective tissue structures, which have been shown to completely replace the SIS material in time.

In this embodiment, SIS is used to temporarily adhere the filter portion 28 to the walls of a body vessel in which the device 10 is deployed. SIS has a natural adherence or wettability to body fluids and connective cells comprising the connective tissue of a body vessel wall. Due to the temporary nature of the duration in which the device 10 is deployed in the body vessel, host cells of the wall may adhere to the filter portion 28 but not differentiate, allowing for retrieval of the device 10 from the body vessel.

In other embodiments, the filter portion may also be made of a mesh cloth, woven nitinol, nylon, polymeric material, teflon, or woven mixtures thereof without falling beyond the scope or spirit of the present invention.

In use, the device 10 expands from the collapsed state to the expanded state, engaging the filter 12 with the body vessel. In turn, the lip 25 of the filter portion 28 expands to open the filter portion 28 for capturing emboli during treatment of the stenotic lesion. After a need for such device 10 in the vasculature passes, the device 10 may be easily retrieved. In one embodiment, a catheter may be used to move longitudinally about the filter 12 to engage and move the struts 14 radially inwardly to collapse the device 10, thereby moving the device 10 toward the collapsed state.

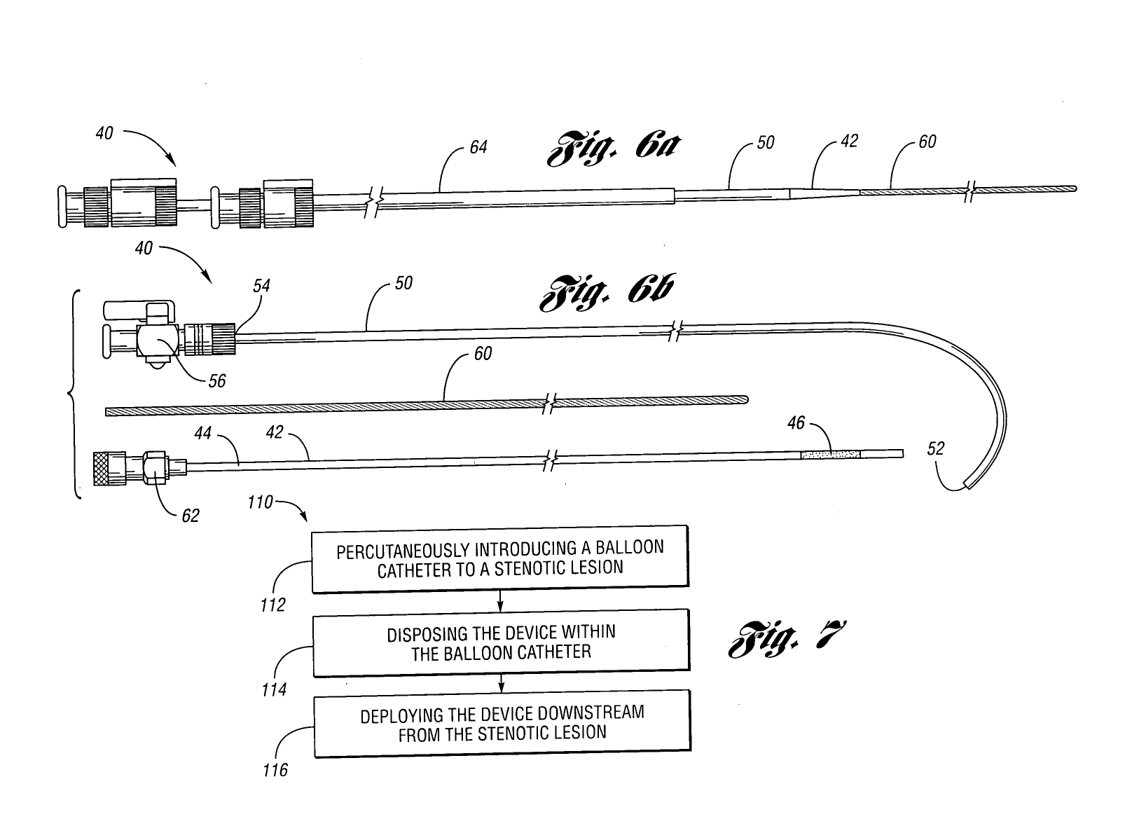

FIGS. 6a and 6b depict an embolic protection assembly 40 for capturing emboli during treatment of a stenotic lesion in a body vessel in accordance with another embodiment of the present invention. As shown, the assembly 40 comprises a balloon catheter 42 having a tubular body 44 and an expandable balloon 46 attached to and in fluid communication with the tubular body 44 for angioplasty at a stenotic lesion. In this embodiment, the assembly 40 comprises the embolic protection device 10 mentioned above. The tubular body 44 is preferably made of soft flexible material such as silicon or any other suitable material. In this embodiment, the balloon catheter 42 may include an outer lumen and an inner lumen. The outer lumen may be in fluid communication with the balloon for inflating and deflating the balloon. The inner lumen may be formed therethrough for percutaneous guidance through the body vessel.

As shown, the assembly 40 further includes an inner catheter 50 having a distal end 52 through which the balloon catheter 42 is disposed for deployment in the body vessel. The inner catheter 50 is preferably made of a soft, flexible material such as silicon or any other suitable material. Generally, the inner catheter 50 further has a proximal end 54 and a plastic adaptor or hub 56 to receive the embolic protection device 10 and balloon catheter 42 to be advanced therethrough. The size of the inner catheter 50 is based on the size of the body vessel in which it percutaneously inserts, and the size of the balloon catheter 42.

As shown, the assembly 40 may also include a wire guide 60 configured to be percutaneously inserted within the vasculature to guide the inner catheter 50 to a location adjacent a stenotic lesion. The wire guide 60 provides the inner catheter 50 (and balloon catheter 42) a path during insertion within the body vessel. The size of the wire guide 60 is based on the inside diameter of the inner catheter 50.

In one embodiment, the balloon catheter 42 has a proximal fluid hub 62 in fluid communication with the balloon via the outer lumen for fluid to be passed therethrough for inflation and deflation of the balloon during treatment of the stenotic lesion. The embolic protection device 10 is preferably coaxially disposed through the inner lumen of the balloon catheter 42 prior to treatment of the stenotic lesion in the body vessel. The distal protection device 10 may be guided through the inner lumen preferably from the hub and distally beyond the balloon of the balloon catheter 42, exiting from the distal end 52 of the balloon catheter 42 to a location within the vasculature downstream of the stenotic lesion.

In this embodiment, the assembly further includes a polytetrafluoroethylene (PTFE) introducer sheath 64 for percutaneously introducing the wire guide 60 and the inner catheter 50 in a body vessel. Of course, any other suitable material may be used without falling beyond the scope or spirit of the present invention. The introducer sheath 64 may have any suitable size, e.g., between about three-french to eight-french. The introducer serves to allow the inner and balloon catheters to be percutaneously inserted to a desired location in the body vessel. The introducer sheath 64 receives the inner catheter 50 and provides stability to the inner catheter 50 at a desired location of the body vessel. For example, the introducer sheath 64 is held stationary within a common visceral artery, and adds stability to the inner catheter 50, as the inner catheter 50 is advanced through the introducer sheath 64 to a dilatation area in the vasculature.

When the distal end 52 of the inner catheter 50 is at a location downstream of the dilatation area in the body vessel, the balloon catheter 42 is inserted therethrough to the dilatation area. The device 10 is preferably loaded through the proximal end of the balloon catheter 42 to a location therein adjacent the expandable balloon 46. The balloon catheter 42 is then advanced through the inner catheter 50 for deployment through its distal end 52. In this embodiment, when the device 10 is passed through the dilatation area, the device may be deployed downstream of the stenotic lesion.

It is understood that the assembly described above is merely one example of an assembly that may be used to deploy the embolic protection device in the body vessel. Of course, other apparatus, assemblies and systems may be used to deploy any embodiment of the embolic protection device without falling beyond the scope or spirit of the present invention.

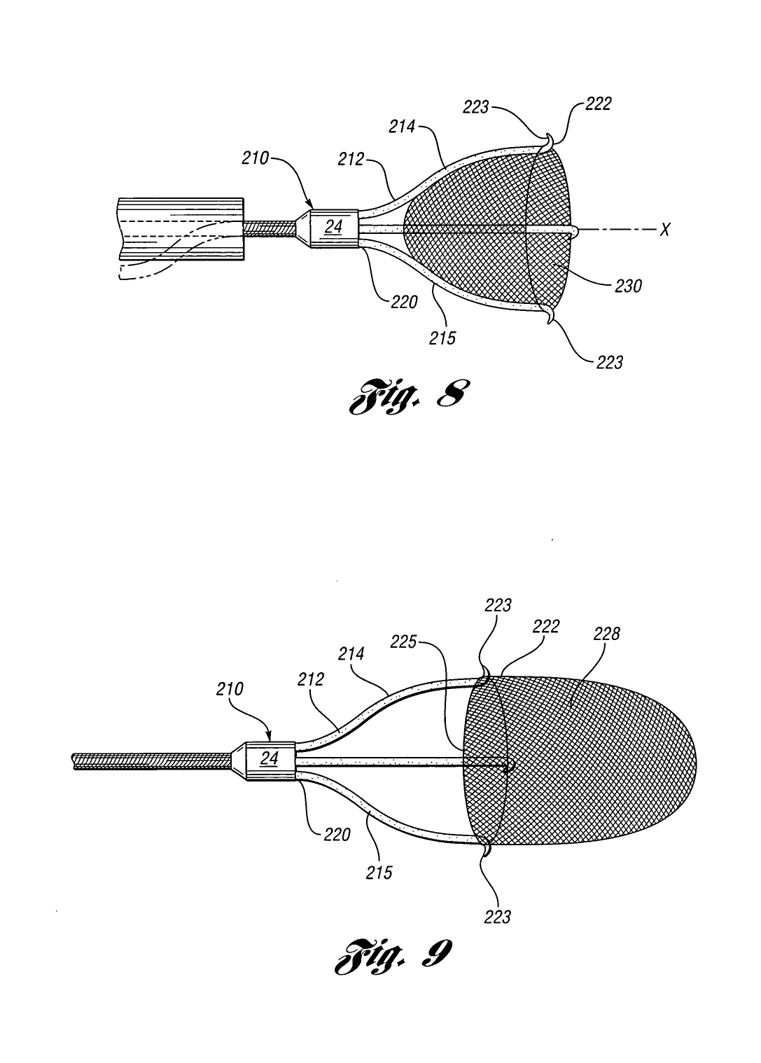

FIG. 7 illustrates a flow chart depicting one method 110 for capturing emboli during treatment of a stenotic lesion in a body vessel, implementing the assembly mentioned above. The method comprises percutaneously introducing a balloon catheter having an expandable balloon for angioplasty of the stenotic lesion in the body vessel in box 112. Introduction of the balloon catheter may be performed by any suitable means or mechanism. As mentioned above, an introducer sheath and a wire guide may be used to provide support and guidance to the balloon catheter. For example, the wire guide may be percutaneously inserted through the introducer sheath to the stenotic lesion in the body vessel. The inner catheter and balloon catheter may then be place over the wire guide for percutaneous guidance and introduction to the stenotic lesion.

The method 110 further comprises disposing the embolic protection device coaxially within the balloon catheter in box 114. The device may be disposed coaxially within the balloon catheter before or after percutaneous insertion of the balloon catheter. For example, once the balloon catheter is placed at the stenotic lesion, the wire guide may be removed therefrom, and the device may then be disposed within the balloon catheter for guidance and introduction in the body vessel. In this example, the expandable balloon is positioned at the stenotic lesion and the device, in its collapsed state, is disposed through the distal end of the balloon catheter downstream from the expandable balloon.

The method 110 further includes deploying the device in a deployed state downstream from the stenotic lesion to capture emboli during treatment of the stenotic lesion in box 116. In the expanded state, the open end of the filter portion is expanded to a proximally facing concave shape for capturing emboli during angioplasty.

The method may further include treating the stenotic lesion in the body vessel with the balloon catheter. In this example, the expandable balloon may be injected with saline and expanded for predilatation. As desired, additional balloon catheters may be used for pre-dilatation treatment, primary dilatation treatment, and post-dilatation treatment of the stenotic lesion while the device is in its expanded state within the body vessel.

FIGS. 8 and 9 illustrate an embolic capture device 210 in accordance with another embodiment of the present invention. As shown, the device 210 comprises components similar to the components of the device 10 shown in FIGS. 2 and 4. For example, the filter 212 including struts 214, arcuate segment 215, first ends 220, second ends 222, and hub 224 of FIGS. 8 and 9 are similar to filter 12 including struts 14, arcuate segment 15, first ends 20, second ends 22, and hub 24 of FIGS. 2 and 4. As shown, each of the second ends 222 has an anchoring hook 223 extending therefrom. In the expanded state, each anchoring hook 223 is configured to engage the wall of a body vessel, thereby lessening the chance of migration of the device 210. The device 210 further comprises a ring 230 that is attached to the second ends 222, allowing the anchoring hooks 223 to distally extend therefrom. In this embodiment, the ring 230 is configured to be collapsible in the collapsed state and expandable in the expanded state of the device 210. This may be accomplished by any suitable manner. For example, the ring may be comprised of superelastic material such as Nitinol, thereby allowing the ring to collapse when the device is collapsed and to expand when the device is expanded.

As shown, the device 210 further comprises a filter portion 228 having a lip 225 circumferentially attached to the ring 230. The filter portion 228 may be attached to the ring by any suitable means including thermal bonding and sonic bonding. The filter portion is configured for allowing blood to flow therethrough and for capturing emboli when the filter 212 is in the expanded state.

The filter 12 may be comprised of any suitable material such as a superelastic material, stainless steel wire, cobalt-chromium-nickel-molybdenum-iron alloy, or cobalt-chrome alloy. It is understood that the filter 12 may be formed of any other suitable material that will result in a self-opening or self-expanding filter, such as shape memory alloys. Shape memory alloys have a property of becoming rigid, that is, returning to a remembered state, when heated above a transition temperature. A shape memory alloy suitable for the present invention may comprise Ni—Ti available under the more commonly known name Nitinol. When this material is heated above the transition temperature, the material undergoes a phase transformation from martensite to austenic, such that material returns to its remembered state. The transition temperature is dependent on the relative proportions of the alloying elements Ni and Ti and the optional inclusion of alloying additives.

In one alternate embodiment, the filter 12 may be made from Nitinol with a transition temperature that is slightly below normal body temperature of humans, which is about 98.6° F. Although not necessarily a preferred embodiment, when the filter 12 is deployed in a body vessel and exposed to normal body temperature, the alloy of the filter 12 will transform to austenite, that is, the remembered state, which for one embodiment of the present invention is the expanded configuration when the filter 12 is deployed in the body vessel. To remove the filter 12, the filter 12 is cooled to transform the material to martensite which is more ductile than austenite, making the filter 12 more malleable. As such, the filter 12 can be more easily collapsed and pulled into a lumen of a catheter for removal.

In another alternate embodiment, the filter 12 may be made from Nitinol with a transition temperature that is above normal body temperature of humans, which is about 98.6° F. Although not necessarily a preferred embodiment, when the filter 12 is deployed in a body vessel and exposed to normal body temperature, the filter 12 is in the martensitic state so that the filter 12 is sufficiently ductile to bend or form into a desired shape, which for the present invention is an expanded configuration. To remove the filter 12, the filter 12 is heated to transform the alloy to austenite so that the filter 12 becomes rigid and returns to a remembered state, which for the filter 12 in a collapsed configuration.

While the present invention has been described in terms of preferred embodiments, it will be understood, of course, that the invention is not limited thereto since modifications may be made to those skilled in the art, particularly in light of the foregoing teaching.

1. An embolic protection device for capturing emboli during treatment of a stenotic lesion in a body vessel, the device comprising:
a filter comprising a plurality of struts having first ends attached together at a center portion along a longitudinal axis, each strut having an arcuate segment extending from the first end to a second end, the struts being configured to move between an expanded state for engaging with the body vessel and a collapsed state for filter retrieval or delivery; and
a filter portion circumferentially attached to the filter at each of the second ends, the filter portion being configured for allowing blood to flow therethrough and for capturing emboli when the filter is in the expanded state.

2. The device of claim 1 further comprising a hub to which the first ends attach at the center point.

3. The device of claim 1 wherein the filter portion includes a lip attached to each of the second ends of the struts defining an opening of the filter portion when the filter is in the expanded state, the lip extending to a closed end for capturing emboli.

4. The device of claim 1 wherein the filter portion is made of small intestine submucosa.

5. The device of claim 1 wherein the filter is made of shape memory material.

6. The device of claim 5 wherein the shape memory material is nitinol.

7. The device of claim 1 wherein each of the second ends of the struts is configured to engage with the body vessel to anchor the device thereto.

8. An embolic protection assembly for capturing emboli during treatment of a stenotic lesion in a body vessel, the assembly comprising:
a balloon catheter having a tubular body portion and an expandable balloon attached to and in fluid communication with the tubular body portion for angioplasty at the stenotic lesion, the expandable balloon having distal and proximal portions; and
an emboli protection device coaxially disposed within the balloon catheter during treatment of the stenotic lesion in the body vessel, the device comprising:
a filter comprising a plurality of struts having first ends attached to each other at a center point along a longitudinal axis, each strut having an arcuate segment extending from the first end to a second end, the struts being configured to move between an expanded state for engaging with the body vessel and a collapsed state for filter retrieval or delivery; and
filter portion circumferentially attached to the filter at each of the second ends, the filter portion being configured for allowing blood to flow therethrough and for capturing emboli when the filter is in the expanded state.

9. The assembly of claim 8 wherein the balloon catheter includes an outer lumen and an inner lumen, the outer lumen being in fluid communication with the balloon for inflating and deflating the balloon, the inner lumen being formed therethrough for percutaneous guidance through the body vessel.

10. The assembly of claim 8 further comprising:
an inner catheter having a distal end throughwhich the balloon catheter is disposed for deployment in the body vessel;
a wire guide configured to be disposed through the inner lumen of the balloon catheter for percutaneous guidance through the body vessel; and
an introducer sheath throughwhich the inner catheter is inserted for percutaneous insertion in the body vessel.

11. The assembly of claim 8 wherein the inner catheter further includes a proximal end, the proximal end having a control handle in fluid communication with the balloon for fluid to be passed therethrough for inflation and deflation of the balloon during treatment of the stenotic lesion.

12. The assembly of claim 8 further comprising a hub to which the first ends attach at the center point.

13. The assembly of claim 8 wherein the filter portion includes a lip attached to each of the second ends of the struts defining an opening of the filter portion when the filter is in the expanded state, the lip extending to a closed end for capturing emboli.

14. The assembly of claim 8 wherein the filter portion is made of small intestine submucosa.

15. The assembly of claim 8 wherein the filter is made of shape memory material.

16. The assembly of claim 15 wherein the shape memory material is nitinol.

17. The assembly of claim 8 wherein each of the second ends of the struts is configured to engage with the body vessel to anchor the device thereto.

18. A method for embolic protection during treatment of a stenotic lesion in a body vessel, the method comprising:
percutaneously introducing a balloon catheter in a body vessel, the balloon catheter having a tubular body portion and an expandable balloon attached to and in fluid communication with the tubular body portion for angioplasty at the stenotic lesion;
disposing an embolic protection device in an undeployed state coaxially within the balloon catheter, the device comprising:
a filter comprising a plurality of struts having first ends attached to each other at a center point along a longitudinal axis, each strut having an arcuate segment extending from the first end to a second end, the struts being configured to move between an expanded state for engaging with the body vessel and a collapsed state for filter retrieval or delivery;
a filter portion circumferentially attached to the filter at each of the second ends, the filter portion being configured for allowing blood to flow therethrough and for capturing emboli when the filter is in the expanded state; and
deploying the device in a deployed state downstream from the stenotic lesion to capture emboli during treatment of the stenotic lesion.
